(19) United States
(12) Patent Application Publication
(10) Pub. No.: US 2024/0181179 A1
(43) Pub. Date: Jun. 6, 2024

(54) SYSTEM AND METHOD FOR VISUALIZATION OF A SURGICAL SITE DURING A MEDICAL PROCEDURE

(71) Applicant: Gilbert Eduardo Flores, San Luis Obispo, CA (US)

(72) Inventor: Gilbert Eduardo Flores, San Luis Obispo, CA (US)

(21) Appl. No.: 18/300,886

(22) Filed: Apr. 14, 2023

Related U.S. Application Data
(60) Provisional application No. 63/430,191, filed on Dec. 5, 2022.

(51) Int. Cl. A61M 13/00

(52) U.S. Cl. CPC A61M 13/003; A61B 17/00234

(57) ABSTRACT

An apparatus and method for improving visualization during medical or surgical procedures by using gas under pressure to disperse debris at the site of the procedure. The debris can be blood, exudates, solid substances, and the like. The disclosed apparatus uses gas under pressure that is focused on the site of the procedure. The disclosed apparatus can be used with or without a medical device or instrument or in designs that incorporate both.

### CROSS-REFERENCE TO RELATED APPLICATIONS

This application claims priority from a U.S. provisional patent application Ser. No. 63/430,191, filed on Dec. 5, 2022, which is incorporated herein by reference in its entirety.

### FIELD OF INVENTION

The present invention relates to a medical device, and more particularly, the present invention relates to an apparatus and method for dispersing debris from a surgical site using gas under pressure to improve visualization.

### BACKGROUND

During medical procedures, the bodily tissues being accessed must be clearly visible. In surgeries, visualization of the surgical area during a procedure is critical. Bodily fluids envelop the tissue and organs hindering their visualization and so must be removed to perform the procedures safely and effectively. Fluids must be removed continuously from the site of interest using one or more of the available options. Camera technology and image resolution have improved aiding visualization; however, the available camera technology cannot help if bodily fluids like blood, plasma, bile, exudate, or any other bodily fluids are obstructing visualization. Mostly, suction, irrigation, or a surgical sponge are used to improve visualization.

The known methods, however, suffer from several shortcomings such as a dry field and the tissue may be too delicate for suction (as in neurosurgery). Irrigation may hinder visualization due to the pooling of fluid. Irrigation may hinder the dissection and cauterization of tissue. Suction may damage the tissue or vessels. Suction and irrigation may not be used at the same time as instruments in the same area as the suction or irrigation cannula gets in the way (this causes pauses of suction/irrigation and thus visualization suffers). Many times, suction may be helpful but draws plasma, bile, exudate, or any other bodily fluids toward the subject area potentially hindering visualization. Suction can also potentially grab on and/or damage delicate tissue. Suction, irrigation, or a surgical sponge are also typically used as separate steps during a procedure. When a patient has blood or debris accumulating quickly, it becomes difficult to stop, clear the area of debris (in a separate step) and then get the surgical instrument back in position before additional blood or bodily debris has accumulated and once again obstructed visualization. This is commonly seen in laparoscopy where the suction device must replace a working instrument due to limited access ports.

A need is therefore appreciated for better visualization tools that overcome the aforesaid drawbacks with the known technologies.

Hereinafter, the terms “gas” and “air” are interchangeably used. The gas can be pure or a mixture of two or more gases. Moreover, the gas can include any chemicals or medicines. Also, the gas under pressure can be at room temperature or any other temperature. The gas under pressure can be in the form of a stream or jet.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an apparatus and method for removing or displacing bodily fluids from a site of the incision/procedure/surgery so that visualization is improved.

It is another object of the present invention that the apparatus and method clear or disperses debris, fluids, smoke, and steam, from a surgical or procedure field.

It is still another object of the present invention that the apparatus and method do not interfere with medical instruments.

It is yet another object of the present invention that the apparatus and method provide a clear view of a surgery site.

It is a further object of the present invention that the apparatus and method can be used with other surgical tools or instruments.

It is still a further object of the present invention that the apparatus can be used simultaneously with a surgical tool or instrument.

It is still a further object of the present invention that the apparatus can be used in designs that combine a surgical tool or instrument and the mentioned apparatus in one.

In one aspect, disclosed is a method for visualization of bodily tissues and organs, in which gas under pressure is directed towards the incision or surgical site to expel or disperse the debris.

In one aspect, disclosed is a method for clearing debris from a surgery site in a medical procedure, the method comprises blowing gas under pressure at the surgery site to disperse the debris or fluid. The method further comprises controlling the rate of flow of the gas under pressure. The method further comprises changing the flow of the gas under pressure from continuous to pulsation mode.

In one aspect, disclosed is a medical apparatus for clearing debris from a surgery site in a medical procedure, the apparatus comprises a functional head; an actuation mechanism for actuating the functional head; a nozzle coupled in proximity to the functional head; a tubing connecting the nozzle to a source of gas under pressure; and a control means for controlling parameters of the gas under pressure. The functional head could be a hook, dissector, grasper, or a wide variety of common tools/instruments for laparoscopy or any and all other medical procedures. The nozzle is positioned such that the gas under pressure is blown over, along, or through the functional head. The control means is configured to start and stop the flow of gas under pressure. The control means is configured to change the flow rate of the gas under pressure. The control means is configured to change the pattern between continuous and pulsation of the gas under pressure.

In one aspect disclosed is a medical apparatus with no functional head wherein the nozzle is positioned such that the gas under pressure is blown from a tip of the medical apparatus.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a schematic view of the medical apparatus having a replaceable cartridge for air supply, according to an exemplary embodiment of the present invention.

FIG. 2 is a schematic view of another implementation of the apparatus, according to an exemplary embodiment of the present invention.

FIG. 3 is an enlarged view showing the nozzle of the apparatus, according to an exemplary embodiment of the present invention.

FIG. 4 is an enlarged view showing the head of the medical apparatus having a laparoscopic functional head, according to an exemplary embodiment of the present invention

FIG. 5 is a block diagram of the apparatus, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

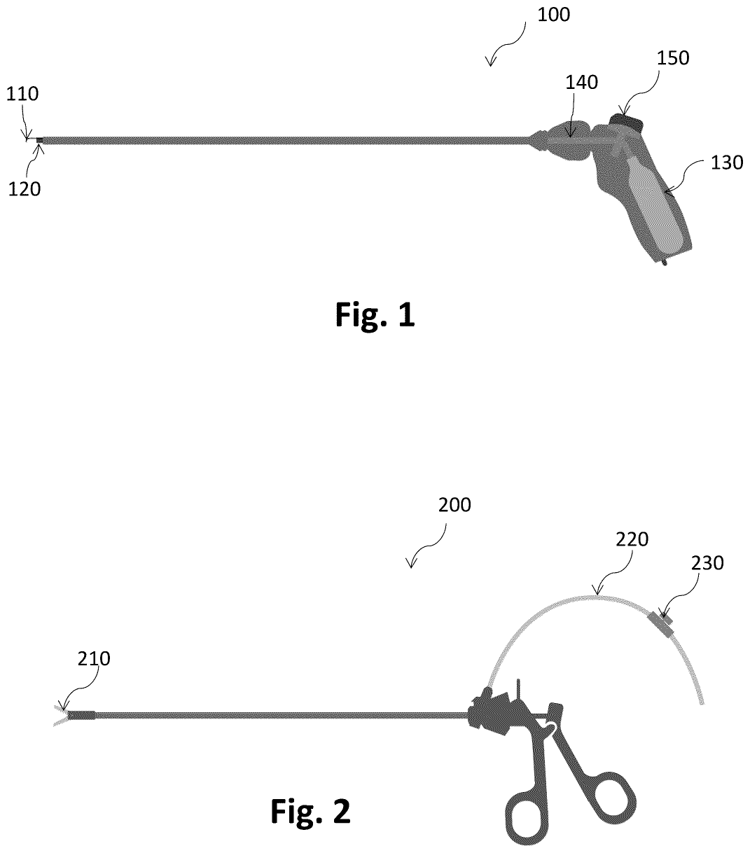

Disclosed are an apparatus and method for supplying gas under pressure to the site of interest in a medical procedure for expelling debris including liquid, solids, semi-solids, and smoke. The debris can include blood, plasma, bile, exudate, or any other bodily fluids that may hinder visualization. Referring to FIG. 1 shows a medical apparatus according to an exemplary embodiment of the present invention. It is understood, however, that the disclosed apparatus can be used independently of the medical device. Moreover, the disclosed device can be used interchangeably or in conjunction with different medical devices.

The disclosed apparatus can include a pressure source for supplying the gas under pressure. The pressure source can be an air compressor that can supply compressed gas. Alternatively, the pressure source can be a tank in which compressed air is stored. Other examples of pressure sources include surgical insufflators, electric compressors, disposable cylinders, and the like. A control mechanism can be used to control the desired air pressure effect. A tubing can be used to carry the compressed air, wherein one end of the tubing can be connected to the pressure source while another end can be connected to a nozzle. The nozzle can be used to direct or focus the gas under pressure. The nozzle can be coupled to a medical device. The nozzle can be interchangeably coupled to the medical device. The medical device can be any surgical tool or instrument that is used to perform a surgical procedure wherein the gas under pressure aids in the visualization of the site. The nozzle can be positioned such that the gas under pressure can be discharged at or near the front of the tool.

In certain implementations, disclosed is a medical apparatus having the medical device, the nozzle, the tubing, control valve and the pressure source. FIG. 1 shows an exemplary embodiment of the medical apparatus 100 that has a functional head 110. For example, the functional head can be a surgical hook. Surgical hooks as used in different laparoscopic procedures can be incorporated. Different kinds of surgical instruments can be interchangeably coupled to the medical apparatus. An actuation mechanism to operate the surgical instrument is known in the art and any such actuation mechanism can be incorporated into the medical apparatus. Different types of functional heads and actuation mechanisms are within the scope of the present invention.

FIG. 1 shows the medical apparatus having a nozzle 120 positioned in proximity to the functional head. The nozzle can focus the gas toward the functional head so that focused gas can remove the debris from the site on which the functional head is working. Tubing 140 can connect the pressure source 130, which is a reservoir, to the nozzle 120. The gas under pressure can be directed in real-time towards the area of surgical interest where blood, plasma, bile, exudate, or any other bodily fluids might be hindering visualization. As the focalized gas clears debris (plasma, bile, exudate, or any other bodily fluids) from the area of interest by displacement, the surgeon has instant and constant improved visualization of the area where the tool can be used.

The disclosed apparatus can be used as a standalone device or can be incorporated into common surgical instruments. Incorporated in a surgical tool can have the advantage that an extra step of clearing debris with a separate tool is unnecessary. Improved visualization greatly improves the safety of all surgical procedures. For example, the disclosed apparatus can be used with or can be incorporated into various laparoscopic instruments, robotic tools, dissectors, clamps, graspers, cautery instruments, and the like. Embodiments of the disclosed apparatus and method can be used in various medical or surgical procedures including laparoscopy, endoscopy, robotic surgery, and open procedures.

The disclosed apparatus may also include control means to control the flow of gas under pressure. Different parameters for the gas under pressure can be controlled/manipulated for desired results. Control means can include options to turn on and off the gas supply, to control the rate of flow, and the pressure of the gas. Suitable hardware to control the different parameters can be provided. For example, a switch can be provided to turn the gas supply on and off. A knob or variable valve can be used to change the flow rate of gas under pressure. For example, FIG. 1 shows button 150 near the handle which can be used to control the flow of gas under pressure. The button is within reach so that while holding the medical apparatus in hand in an operating state, the button can be actuated by a finger or thumb of the hand. FIG. 2 shows another embodiment of the medical apparatus 200 that has a dissector 210 as the functional head, a tubing 220, and a control valve 230 is shown away from the handle interrupting the tubing.

The control means can also provide for changing the airflow pattern, such as a continuous stream of air, air flow in pulses, and the like. The air or gas pressure/pulsation or constant flow can be precisely controlled with the control means. Besides the control means, the gas discharge pattern can also be modified by changing the effective size of the opening of the nozzle.

The disclosed apparatus can be advantageous in that it can be used at the same time as dissection, cauterization, and suturing without the need for interruption as when suction or irrigation is used. It does not hinder visualization of the area of interest as with suction or irrigation. It causes less traumatic effects to delicate tissues than with suction. The disclosed apparatus does not cause a fluid pooling effect as with irrigation which can affect cauterization, dissection, and visualization. The disclosed apparatus can be used in any medical or surgical procedure including laparoscopy, endoscopy, robotic surgery, and open procedures.

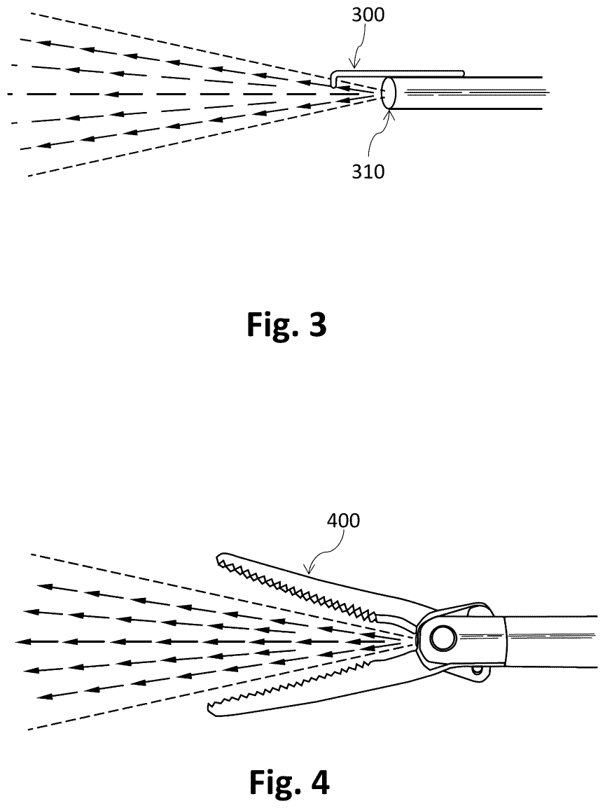

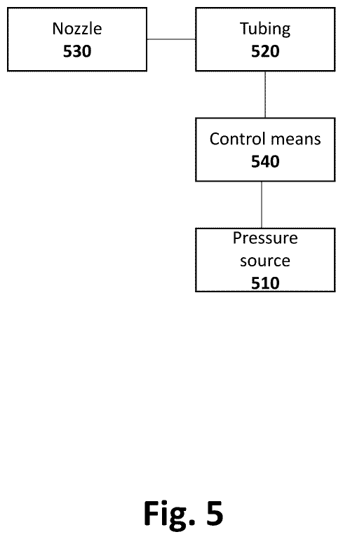

FIG. 3 is an enlarged view showing a hook 300 and a nozzle 310. FIG. 4 shows an enlarged view of the front of a medical apparatus having a dissector as the functional head 400 and the nozzle is positioned in center of the functional head. FIG. 5 is a block diagram showing a pressure source 510, a tubing 520, a nozzle 530, and a control means 540.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

1. A method for cleaning debris from a surgery site to improve visualization in a medical procedure, the method comprises:
blowing gas under pressure at the surgery site to disperse the debris, the gas is pure or a mixture of gases.

2. The method according to claim 1, wherein the method further comprises:
controlling a rate of flow of the gas under pressure.

3. The method according to claim 1, wherein the method further comprises:
changing a flow of the gas under pressure from continuous to pulsation mode.

4. A medical apparatus for cleaning debris from a surgery site or incision to improve visualization in a medical procedure using a directed or focalized gas under pressure allowing instant and constant improved visualization, wherein the apparatus comprises:
a functional head;
an actuation mechanism for actuating the functional head;
a nozzle coupled in proximity to the functional head;
a tubing connecting the nozzle to a source of gas under pressure; and
a control means for controlling one or more parameters of the gas under pressure, wherein the one or more parameters comprises rate of flow and pressure,
wherein the gas under pressure is configured to be directed towards the surgical site or incision for expelling or dispersing the debris,
wherein the medical apparatus cannot heat the tissues.

5. The medical apparatus according to claim 4, wherein the functional head is a hook for laparoscopy procedure.

6. The medical apparatus according to claim 4, wherein the functional head is a dissector for laparoscopy procedure.

7. The medical apparatus according to claim 4, wherein the functional head is a grasper for laparoscopy procedure.

8. The medical apparatus according to claim 4, wherein the nozzle is positioned such that the gas under pressure is blown over and along the functional head towards the surgical site.

9. The medical apparatus according to claim 4, wherein the nozzle is positioned such that the gas under pressure is blown through the functional head towards the surgical site.

10. The medical apparatus according to claim 4, wherein the apparatus is configured to allow readjusting a position and/or angle of the nozzle relative to the apparatus.

11. The medical apparatus according to claim 4, wherein the control means is configured to start and stop a flow of the gas under pressure.

12. The medical apparatus according to claim 11, wherein the control means is configured to change flow rate of the gas under pressure.

13. The medical apparatus according to claim 12, wherein the control means is configured to change a pattern between continuous flow of the gas under pressure and pulsation of the gas under pressure.

14. A medical apparatus for cleaning debris from a surgery site or incision to improve visualization in a medical procedure using a directed or focalized gas under pressure allowing instant and constant improved visualization, wherein the apparatus comprises:
a nozzle;
a tubing connecting the nozzle to a source of gas under pressure; and
a control means for controlling one or more parameters of the gas under pressure, wherein the gas under pressure is configured to be directed towards the surgical site or incision for expelling or dispersing the debris, wherein the one or more parameters comprises rate of flow and pressure.

15. The medical apparatus according to claim 4, wherein the debris comprises bodily fluids, the bodily fluids comprise blood, plasma, bile, and exudate.

16. The medical apparatus according to claim 4, wherein the one or more parameters are changed by changing an effective size of an opening of the nozzle.

17. The medical apparatus according to claim 4, wherein the nozzle is positioned such that the gas under pressure is blown from a tip of the medical instrument.

18. The medical apparatus according to claim 4, wherein the nozzle is positioned such that the gas under pressure is discharged at or over a front of the medical apparatus.
